(19) United States
(12) Patent Application Publication
(10) Pub. No.: US 2007/0048239 A1
(43) Pub. Date: Mar. 1, 2007

(54) Cosmetic and Personal Care Formulations with Goniochromatic Non-Quarter Wave Multi-Quadrant Multi-Layer Effect Materials

(75) Inventors: Leila S. Song, Putnam Valley, NY (US); Betty Aucar, Ossining, NY (US); Gabriel Uzunian, Rye, NY (US)

Correspondence Address: BASF CATALYSTS LLC, 101 WOOD AVENUE, ISELIN, NJ, 08830, US

(73) Assignee: Engelhard Corporation, Iselin, NY (US)

(21) Appl. No.: 11/422,268

(22) Filed: Jun. 5, 2006

Related U.S. Application Data
(60) Provisional application No. 60/711,758, filed on Aug. 26, 2005.

(51) Int. Cl. A61K 8/19

(52) U.S. Cl. 424/63; 977/926

(57) ABSTRACT

Cosmetic and personal care formulations with goniochromatic non-quarter wave multi-quadrant multi-layer effect materials includes a transparent substrate such as borosilicate, a layer of high refractive index material on the substrate, and alternating layers of low refractive index and high refractive index materials on the first layer, the total number of layers being an odd number of at least three, all adjacent layers differing in refractive index by at least about 0.2 and at least one of the layers having an optical thickness which is different from all of the other layers. The resulting multilayer effect pigment is not a quarter-wave stack. Further color intensity, dimension and depth is obtained by combining the goniochromatic effect material with combination pigment which is obtained by combining a transparent substrate, an interference pigment and an absorption colorant.

This application claims priority to pending U.S. Provisional patent application No. 60/711,758 filed Aug. 26, 2005 incorporated herein in its entirety.

### FIELD OF THE INVENTION

This invention relates to cosmetic and personal care formulations. In particular, this invention relates to cosmetic formulations having brilliant, intense goniochromatic multi-quadrant interference color travel effect.

### BACKGROUND OF THE INVENTION

Effect pigments, also known as pearlescent or nacreous pigments, are based on the use of a laminar substrate such as mica or glass flake, which has been coated with a metal oxide layer. These pigments exhibit pearl-like luster as a result of reflection and refraction of light, and depending on the thickness of the metal oxide layer, they can also exhibit interference color effects.

Titanium dioxide-coated mica and iron oxide-coated mica effect pigments are the effect pigments which are encountered most often on a commercial basis. Pigments in which the metal oxide has been over-coated with another material are also well known in the art.

The commercially available effect pigments which contain only a single coating of a high refractive index material provide only two reflecting interfaces between materials. These two material interfaces (and reflections) are therefore solely responsible for the reflectivity achieved from the platelet surface. A substantial percentage of the incident light is thus transmitted through the platelet and while this is necessary to create the nacreous appearance of the pigment, it also diminishes other desirable properties of the effect pigments such as luster, chromaticity and hiding power. To counteract this consequence, the art has either mixed the effect pigments with other pigments or added additional layers of transparent and/or selectively absorbing materials onto the effect pigment.

Examples of prior art describing multi-coated effect pigments include JP 7-246366, WO 98/53011, WO 98/53012 and U.S. Pat. No. 4,434,010. All of such prior art requires that each coated layer possess an optical thickness equal to a whole number multiple of a one-quarter of the wave-length at which interference is expected. Such construction of the so-called quarter-wave stacks is a widely accepted and implemented condition in the thin-film industries. Because of this limitation, a unique layer thickness combination is essential in order to create each individual one of the interference colors of the visible spectrum. The base substrate is the only dimension common to all of the compositions displaying different interference colors.

It has been discovered that the adherence to the quarter-wave stack approach is unnecessary and suitable products, even with substantial gains in luster, chromaticity and hiding power, can be achieved without observing that requirement. Further, numerous other advantages can be realized.

Conventionally, it was essential to use transparent, translucent and semi-opaque cosmetic base formulations in order to achieve the best visible goniochromatic travel effect for cosmetic applications. However, it has now been discovered that visible goniochromatic interference color travel effect can be produced in cosmetic formulations having opaque bases by using goniochromatic non-quarter-wave, multi-layer interference effect pigments containing a borosilicate substrate.

The goniochromatic interference color travel effect can be produced and intensified when the goniochromatic non-quarter-wave, multi-layer interference effect materials are blended with effect pigments such as combination pigments having both absorption and reflection colors.

It is an aspect of this invention to provide brilliant, intense goniochromatic interference color travel effect material for use in cosmetic and personal care formulations.

### SUMMARY OF THE INVENTION

This invention relates to cosmetic and personal care formulations. In particular, this invention relates to cosmetic formulations having brilliant, intense goniochromatic interference color travel effect. The formulations use non-quarter wave, multi-layer goniochromatic color travel effect pigments. Optionally, such pigments may be combined with multi-layer effect/combination pigments to produce and intensify the color travel effect.

### BRIEF DESCRIPTION OF THE DRAWING

FIG. 1 is a graph comparing the color travel of a pigment of this invention with a commercial pigment.

FIG. 2 is a graph comparing the color travel of a cosmetic containing the inventive pigment with a cosmetic containing a commercial pigment.

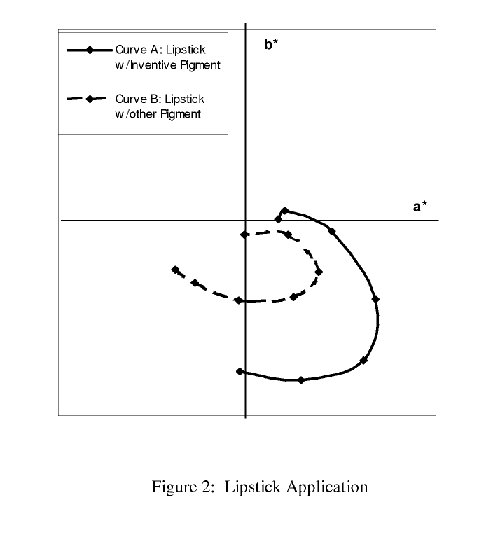

### DESCRIPTION OF THE INVENTION

In accordance with the present invention, the cosmetic and personal care formulations include a non-quarter-wave multi-layered effect pigment product composed of a transparent substrate having an odd number of layers thereon and in which at least one of the layers has an optical thickness which is different from all of the other layers causing the pigment not to be a quarter-wave stack, i.e., non-quarter wave pigment. The non-quarter wave goniochromatic pigments of this invention are described in commonly assigned pending patent application Ser. 60/652020 filed Feb. 12, 2005 incorporated in its entirety herein by reference. Multi-layer effects pigments are also described in commonly assigned U.S. Pat. No. 6,875,264 and US Patent Application Publication 2005/0166799 incorporated in their entireties herein by reference.

Any encapsulatable smooth and transparent platelet can be used as the substrate to make the non-quarter-wave multi-layered effect pigment. Examples of useable platelets include mica, whether natural or synthetic, kaolin, glass flakes, borosilicate, bismuth oxychloride, platy aluminum oxide, or any transparent platelet of the proper dimensions. Pigments containing borosilicate-based substrates are pure (free from impurities), smooth (little light scattering) and with high transparency and chroma. The substrate need not be totally transparent but should, preferably, have at least about 75% transmission. The size of the platelet shaped substrate is not critical per se and can be adapted to the particular use. Generally, the particles have major dimensions averaging about 5-250 microns, preferably 5-100 microns, and an aspect ratio greater than about 5. The specific free surface area (BET) of the substrate is, in general, from about 0.2 to 25 m²/g.

The layers encapsulating the substrate alternate between high refractive index materials and low refractive index materials. The high refractive index materials can be anatase titanium dioxide, rutile titanium dioxide, iron oxide, zirconium dioxide, zinc oxide, zinc sulfide, bismuth oxychloride or the like. The CRC Handbook of Chemistry and Physics, 63^{rd} Edition reports refractive indices for these high refractive index materials as follows.

| Material | Refractive Index |
|---|---|
| TiO₂-anatase | 2.55 |
| TiO₂-rutile | 2.90 |
| Fe₂O₃-hematite | 3.01 |
| ZrO₂ | 2.20 |
| ZnO | 2.03 |
| ZnS | 2.38 |
| BiOl | 2.15 |

The low refractive index material can be silicon dioxide, magnesium fluoride, aluminum oxide, a polymer such as polymethyl methacrylate, polystyrene, ethylene vinyl acetate, polyurea, polyurethane, polydivinyl benzene and the like. The CRC handbook of Chemistry and Physics, 63^{rd} Edition reports refractive indices for these low refractive index materials as follows.

| Material | Refractive Index |
|---|---|
| SiO₂-amorphous | 1.46 |
| MgF₂ | 1.39 |
| A1₂O₃ | 1.76 |
| Polymers | 1.4-1.6 is typical |

Any combination of materials can be selected provided that adjacent layers differ in refractive index by at least about 0.2, and more preferably at least about 0.6. The materials are transparent but may, like iron oxide, have an absorption component.

The phrase “a layer of titanium dioxide on said transparent substrate (i) or said optional coating (ii)” as used herein means that the titanium dioxide may be in direct contact with the transparent substrate or an optional coating may be present between the transparent substrate and the layer of titanium dioxide or additives may be present between the transparent substrate and the titanium dioxide layer. The phrase “a subsequent layer of a low refractive index material on said titanium dioxide layer” as used herein means that the subsequent layer of a low refractive index material may be in direct contact with the titanium dioxide layer or additives or other layers may be present between the subsequent layer of a low refractive index material and the titanium dioxide layer. The phrase “an outermost layer of a high refractive index material placed on said subsequent layer (iv) or said optional coating (v)” as used herein means that the outermost layer of a high refractive index material may be in direct contact with the subsequent layer or the optional coating may be present between the outermost layer of a high refractive index material and the subsequent layer or additives may be present between the outermost layer of a high refractive index material and the subsequent layer.

The individual layers can be applied to the substrate and to each other using techniques well known in the art. Any such technique can be utilized. One of the advantages of non-quarter-wave effect pigments is that sol-gel techniques can be used to apply the coatings. Such techniques are well known and widely practiced for thin film deposition, and are safe, economical and amenable to a wide variety of particle shapes and sizes. Chemical vapor deposition techniques which have been used in some prior art have a litany of negative aspects including safety hazards, expensive reagents and infrastructure and substrate particle size limitations. Monolithic web-based multi-layer coating techniques have also been used in the prior art and suffer from the disadvantages that pigment particles are formed after the coatings are applied and therefore have discontinuities in the layers at the fracture points. The particles must also be classified according to size after the monolith is fractured, whereas here the particle size can be predetermined before the coating and can be constant. Useful additives include rutile directors for titanium dioxide such as tin.

Another advantage of non-quarter-wave effect pigments is that the substrate and all layers have an appreciable degree of transparency and therefore the resulting pigments can exhibit unique angle dependent reflectivity ranging from nearly totally reflecting to substantially transmitting as the viewing angle is changed. Many multi-coated pigments in the prior art use metal flakes as substrates and such metal layers are not capable of transmitting light and the resulting pigment is therefore totally opaque.

Because the non-quarter-wave effect pigment is not a quarter-wave stack, the first layer which is adjacent the substrate can be given a fixed thickness and by varying the thickness of the other layers, it is possible to prepare all of the interference colors desired. Further, the first and second coating layers may be fixed and such coated substrates may be used to prepare multiple final products by variation of the final layer only. The number of unique layer combinations necessary to prepare all of the interference colors with the above-described invention is much less than for the prior art. The adherence to the quarter-wave optical thickness condition for the layers of the prior art compositions precludes the use of universal single or double coated precursors to three layer compositions.

While any odd number of layers equal to or greater than three can be employed, it has been found that substantial advantages are present when there are three layers and this is therefore preferred.

The low refractive index material is preferably silica and while this can have other thicknesses, the silica layer preferably has a thickness of at least 100 nm, preferably in the range of about 125-500 nm, and more preferably about 150-320 nm. This maximizes the degree of angle dependent color travel, which is inherent in silica films. Here, the silica layers will have a thickness to provide a variable pathlength for light dependent on the angle of incidence of light impinging thereon. It is preferred that the low refractive index material layer have a sufficient thickness to provide at least more than 75 and, more preferably, more than 100 degrees of hue angle color travel.

The first layer on the substrate and the outermost layer can be the same or different, and are further preferably titanium dioxide. Prior to the formation of the titanium dioxide layer, the substrate may have an optional coating thereon. The optional coating may be a metal oxide such as SiO₂ or a rutile director such as tin. It will be appreciated that where the first or innermost layer has a fixed thickness and the low refractive index layer also has a predetermined thickness, the outermost high refractive index layer will control the interference color as a result of its thickness. The substrate/first layer/subsequent layer combination thus acts as a universal base from which all interference colors can be realized by simply varying the thickness of the third layer. In general, it is useful to provide a first layer of titanium dioxide on the substrate, which will lead to a preliminary white-colored material. As such, the thickness of the first titanium dioxide layer will generally range from about 45 to 65 nm.

The thickness of the third layer, when it is titania, in such an arrangement generally varies from about 20 to 100 nm, and preferably about 40-100 nm. More consistent color can be achieved if the outermost titania layer is at least 40 nm. Here, the pigments of this invention have non-white hues. A “non-white” hue according to this invention means the pigments of this invention will have a chromaticity (0 degrees C*) of at least 40.0 and are not a white to pearl or silvery color.

In the cosmetic and personal care field, these pigments can be used in the eye area, lip area and in all external and rinse-off applications. Thus, they can be used in hair sprays, face powder, leg-makeup, insect repellent lotion, mascara cake/cream, nail enamel, nail enamel remover, perfume lotion, and shampoos of all types (gel or liquid). In addition, they can be used in shaving cream (concentrate for aerosol, brushless, lathering), skin glosser stick, skin makeup, hair groom, eye shadow (liquid, pomade, powder, stick, pressed or cream), eye liner, cologne stick, cologne, cologne emollient, bubble bath, body lotion (moisturizing, cleansing, analgesic, astringent), after shave lotion, after bath milk and sunscreen lotion. Moreover, the non-quarter wave pigments can be used in lipsticks, lip gloss, etc.

In general, the visible goniochromatic multi-quadrant interference color travel effects would be diminished by the opacity of the cosmetic and personal care products with opaque bases. The strong and intense color travel effect of the goniochromatic non-quarter-wave multi-quadrant interference color travel effect pigment enable the pigments to be used in the cosmetic and personal care products with an opaque base. Visible goniochromatic multi-quadrant interference color travel effect can be produced in formulations having opaque bases by using goniochromatic non-quarter-wave multi-layer multi-quadrant interference color travel borosilicate effect pigment. The goniochromatic multi-quadrant interference color travel effect can be produced and intensified when the goniochromatic non-quarter-wave multi-layer multi-quadrant interference effect materials are blended with effect pigments, such as combination pigments, having both absorption and reflection colors, e.g. Durocrome® Iridescent Colors made by Engelhard Corporation, Iselin, N.J. Thus, the goniochromatic non-quarter-wave multi-quadrant interference color travel effect pigments may be used in a wide spectrum of cosmetic and personal care applications. When the cosmetic and personal care products contain the goniochromatic non-quarter-wave multi-quadrant interference color travel effect pigments, the goniochromatic multi-quadrant interference color travel effects can be not only seen in the transparent, translucent, and semi-opaque formulations (e.g. nail enamels, eye gels, hair gels, shampoos, lip glosses, glycerin soaps, etc.), but also in the opaque formulations (e.g. make-up foundations, creams, lotions, etc.)

Those pigments which have an absorption pigment added to an interference pigment resulting in enhanced color intensity are called combination pigments. The addition of absorption pigments to interference pigments enhances reflection colors. In most cases, the absorption pigments have been precipitated onto the interference pigments so that they form an integral part of the platelets. Thus, for example, if Fe₂O₃ which has a yellow to red color depending on its particle size is precipitated upon a yellow interference color, an enhancement of the yellow color will be produced. The yellow of the Fe₂O₃ adds to the yellow of the interference color producing a rich lustrous yellow color.

Other colorants besides Fe₂O₃ have been used. In order to enhance the red interference color, carmine, an organic red colorant, is added to a red interference pigment. In order to enhance the blue, iron blue is added, and in order to enhance the green, Cr₂O₃ is added.

If a colored oxide is used for the coating on mica, that color will combine with the added colorant and will modify the final absorption color. If a colorless oxide is used for the coating on mica, the absorption color will not be modified.

The concentration of the absorption pigments is adjusted so that the color intensity produced is of the same order of magnitude as the interference colors. If the concentration of the colorants is too great, the absorption colorant will obscure the interference color and no enhancement will take place. In order for this enhancement to take place, the colorants are added at a concentration between about 2% and 5% in the case of TiO₂ coated mica pigments, based upon the weight of titanium dioxide coated mica.

Not only can the absorption pigment of the same color as the interference color be added but different absorption colorants can be added to different interferences colors. Thus, for example, it is possible to add a red absorption pigment (carmine) to a blue interference pigment.

When absorption colorants differ in color from the interference color, interesting color effects are produced depending on the background and the angle of viewing. Since the concentration of the colorants is quite low, when the combination pigments are displayed over a black background, the black absorbs the color of the colorant and only the reflection color of the interference is observed. This is true whether the pigments are observed at the normal angle or the grazing angle.

When the combination pigments are dispersed in a film-forming medium and coated over a white background, two distinct colors can be observed depending on the angle of viewing. At the normal angle of viewing, the reflection color of the interference pigment is seen. At the diffuse angle or the grazing angle, the reflection color of the interference pigment is no longer observed and the color of the absorption pigment is now seen. Thus by changing the angle of viewing from the normal to a grazing angle, the color changes from the reflection color of the interference to the color of the absorption pigment. Very beautiful and aesthetically pleasing color effects can be seen.

Combination pigments have been used in applications such as coating on white, grey or black substrates or incorporating them into formulations used for cosmetic applications such as eye shadow, etc. where such color changes would be desirable.

A third color can be seen when the known combination pigments are incorporated into a transparent film-forming medium and coated on a transparent substrate such as glass, acrylic sheet etc. This third color is different in color from both the reflection color and the color of the absorption pigment. The third color is formed from the mixing of the transmission color of the interference pigment with the color of the absorption pigment.

For a well-rounded discussion of combination pigments, (Engelhard Corporation Duochrome®), see U.S. Pat. No. 5,008,143 issued on Apr. 16, 1991 to Armanini. The foregoing reference is hereby incorporated by reference herein for their teachings of combination pigments, processing of combination pigments, demonstrations on types and examples of colors formed.

Cosmetic and personal care formulations having extraordinary goniochromatic multi-quadrant interference color travel effect with visual depth and the appearance of dimensionality can be achieved when using goniochromatic non-quarter-wave, multi-layer, multi-quadrant, interference borosilicate-based effect pigments. These formulations exhibit outstanding luster and interference color properties including goniochromaticity, at levels not possible if traditional single coated pearlescent effect pigments or quarter-wave stacks are used. The important effect created by the formulation with goniochromatic non-quarter-wave multi-layer multi-quadrant interference color travel effect materials is superior to other based effect pigments, in particular, when borosilicate-based substrates are used. Pigments of borosilicate-based substrates are pure (free from impurities), smooth (little light scattering) and have high transparency and chroma. The goniochromatic non-quarter-wave multi-layer multi-quadrant interference color travel effect materials showed stronger color travel than traditional goniochromatic pigments, stronger color travel and chroma than even the mica-based goniochromatic pigments, and showed stronger the effects in opaque system, i.e. opaque lipsticks. The outstanding results were further enhanced when the goniochromatic non-quarter-wave multi-layer multi-quadrant interference borosilicate-based effect pigments were combined with combination pigments.

L*, a*, and b* data are described in Richard S. Hunter, The Measurement of Appearance, John Wiley & Sons, 1987. These CIELab measurements characterize the appearance of the product in terms of its lightness-darkness component, represented by L*, a red-green component represented by a*, and a yellow-blue component represented by b*.

An additional parameter may be derived from the L*, a*, and b* data: the chroma (C) which is [(a*)²+(b*)²]^{1/2}. Chroma refers to the intensity or vividness of the color.

In order to demonstrate the invention, various examples are set forth below. In these examples, as well as throughout this specification and claims, all parts and percentages are by weight and all temperatures are in degrees Centigrade, unless otherwise indicated. In each of the examples, multi-quadrant interference effect material includes a calcium sodium borosilicate substrate, at least one titanium dioxide layer and at least one silica layer. The suitability of multi-quadrant interference effect materials for any particular cosmetic application has not been established, and is the responsibility of the end user.

### EXAMPLE 1

A 5 liter Morton flask was equipped with a mechanical stirrer and charged with a suspension of 150 grams of natural mica of average diameter 50 microns in 1.0 liter of H₂O. The slurry was heated to 74° C. and stirred at 200 RPM and lowered to pH 2.2 with HCl. A 40% TiCl₄ solution was pumped in at 0.75 mls. per minute at pH 2.2 until the mica shade was a white pearl, requiring 190 grams of solution. The pH was kept constant by adding 35% NaOH solution during the addition.

The slurry pH was raised rapidly to 8.25 by adding 35% NaOH solution, and the stirring rate was raised to 250 RPM. 1563.0 grams of 20% Na₂SiO₃.5H₂O solution were added at 5.7 grams/minute while maintaining the pH at 8.25 with 28% HCl solution. A small sample of suspension was then filtered and calcined at 850 degrees C. The interference color of the platelet was yellow as predicted from the titania plus silica film combination.

The suspension pH was then lowered to 2.2 by adding 28% HCl solution at a rate of 0.75 mls/minute. The stirring rate was lowered again to 200 RPM. The second titania layer was coated by again adding 40% TiCl₄ solution at 0.75 mls/minute. A few small samples of suspension were filtered, calcined at 850 degrees C., and evaluated in drawdown until the target product was obtained at 253 grams of added 40% TiCl₄. Thus, the goniochromatic pigment comprised: (i) transparent substrate of mica; (ii) no optional coating on the mica; (iii) a titanium dioxide layer on the mica; (iv) a silica layer on the titanium dioxide layer (iii); (v) no optional coating on the silica; and (vi) a titanium dioxide layer on the silica layer (iv). The entire suspension was then processed to yield the desired calcined product, which exhibited a high chromaticity green normal color which flopped to a violet color at a grazing angle of the drawdown card. The color properties of the pigment agreed with the properties of Sample 19 in the Table of Example 6.

### EXAMPLE 2

A 5 liter Morton flask was equipped with a mechanical stirrer and charged with a suspension of 832 grams of borosilicate glass flake of average diameter 100 microns in 1.67 liters of H₂O. The composition of the borosilicate substrate is shown in the Table.

| Ingredient | Compositions, wt % |
|---|---|
| SiO₂ | 65-72 |
| Al₂O₃ | 1-7 |
| CaO | 4-11 |
| MgO | 0-5 |
| B₂O₃ | 0-8 |
| ZnO | 0-6 |
| R₂O (Na₂O + K₂O) | 9-17 |

The suspension was heated to 80 degrees C., stirred at 300 RPM and adjusted to pH 1.4 with 28% HCl. 47.0 grams of 20% SnCl₄.5H₂O solution were pumped in at 2.4 grams per minute while maintaining the pH at 1.4 with 35% NaOH solution, and then the suspension was stirred for a 30 minute digestion period at temperature.

A 40% TiCl₄ solution was added at 2.0 grams per minute until a white pearl shade was imparted to the glass at 144 grams of added solution. No sample was withdrawn, and the suspension pH was rapidly raised to 8.25 by adding 35% NaOH solution, which was also used to control the pH at 1.4 during the TiCl₄ addition. The temperature was lowered to 74 degrees C., and then 1290.0 grams of 20% Na₂SiO₃.5H₂O solution were added at 5.4 grams per minute while controlling the pH at 8.25 with 28% HCl solution. A small sample of the suspension was filtered and calcined at 625 degrees C.

The suspension pH was lowered to 1.4 with 28% HCl solution added at 0.8 mls/minute, and the temperature was returned to 80 degrees C. The previous SnCl₄.5H₂O addition step was repeated verbatim, as was the 40% TiCl₄ addition. Three samples of the suspension were filtered and calcined at 625 degrees C. after 106 grams, 164 grams and 254 grams of added TiCl₄ solution respectively. The normal interference colors of the 3 samples were blue, turquoise and green which flopped to red, violet and blue-violet respectively at grazing viewing angles. The green normal color sample was essentially an exact analog to the final product yielded in Example 1. All three samples exhibited substantially higher chromaticity than the commercially available singly coated glass flake products (Engelhard Corporation REFLECKS™). The blue pigment had color properties which agreed with Sample 8 of the Table in Example 6. Thus, the goniochromatic pigment comprised: (i) transparent substrate of borosilicate glass flake; (ii) SnO₂ coating on the glass flakes; (iii) a titanium dioxide layer on the SnO₂ coating; (iv) a silica layer on the titanium dioxide layer (iii); (v) SnO₂ coating on the silica layer (iv); and (vi) a titanium dioxide layer on the SnO₂ coating (v).

### EXAMPLE 3

Following the general procedure given in Example 2, a red to yellow color shifting effect pigment was prepared by repeating the first TiO₂ layer white pearl shade of Example 1, adding 860.3 grams of the 20% Na₂SiO₃.5H₂O solution, and a final TiO₂ layer from 293 grams of 40% TiCl₄ solution. The pigment had color properties, which agreed with Sample 3 of the Table of Example 6.

### EXAMPLE 4

Following the general procedure given in Example 2, a violet to orange color shifting effect pigment was prepared by repeating the first TiO₂ layer white pearl shade, adding 1147.0 grams of the 20% Na₂SiO₃.5H₂O solution, and a final TiO₂ layer from 133 grams of added 40% TiCl₄ solution. The pigment had color properties, which agreed with Sample 5 of the Table of Example 6.

### EXAMPLE 5

A 5 liter Morton flask was equipped with a mechanical stirrer and charged with a suspension of 250 grams of borosilicate glass flake of average diameter 81 microns and a BET specific surface area measured at 0.75 m²/gr. in 1.2 liters of H₂O. The suspension was heated to 82° C., stirred at 300 RPM and adjusted to pH 1.4 with 28% HCl. 56.0 grams of 20% SnCl₄.5H₂O solution were pumped in at 2.4 grams per minute while maintaining the pH at 1.4 with 35% NaOH solution, and then the suspension was stirred for a 30 minute digestion period at temperature.

A 40% TiCl₄ solution was added at 2.0 grams per minute until a white pearl shade was imparted to the glass at 173 grams of added solution. No sample was withdrawn, and the suspension pH was rapidly raised to 8.25 by adding 35% NaOH solution, which was also used to control the pH at 1.4 during the TiCl₄ addition. The temperature was lowered to 74 degrees C., and then 1393.8 grams of 20% Na₂SiO₃.5H₂O solution were added at 5.4 grams per minute while controlling the pH at 8.25 with 28% HCl solution. A small sample of the suspension was filtered and calcined at 625 degrees C. and the dry interference color was the same as that of the titania plus silica combination in example 1.

The suspension pH was lowered to 1.4 with 28% HCl solution added at 1.0 mls/minute, and the temperature was returned to 82 degrees C. The previous SnCl₄.5H₂O addition step was repeated verbatim, as was the 40% TiCl₄ addition. Three samples of the suspension were filtered and calcined at 625 degrees C. after 133 grams, 190 grams and 281 grams of added TiCl₄ solution respectively. The normal interference colors of the 3 samples were blue, turquoise and green which flopped to red, violet and blue-violet respectively at grazing viewing angles. The 3 samples were essentially exact analogs to the products yielded in Example 2. Thus, the goniochromatic pigment comprised: (i) transparent substrate of borosilicate glass flake; (ii) SnO₂ coating on the glass flakes; (iii) a titanium dioxide layer on the SnO₂ coating; (iv) a silica layer on the titanium dioxide layer (iii); (v) SnO₂ coating on the silica layer (iv); and (vi) a titanium dioxide layer on the SnO₂ coating (v).

EXAMPLE 6
Effect pigment products on a substrate are set forth in the following table.
Film Thickness and Theoretical Color Data

| Sample | Normal | First TiO₂ | Silica | Second TiO₂ | | | | | | | | |
|---|---|---|---|---|---|---|---|---|---|---|---|---|
| No. | Color² | Layer, Nm¹ | Layer, Nm¹ | Layer, Nm¹ | 0° L* | 0° a* | 0° b* | 0° C* | 60° L* | 60° a* | 60° b* | 60° C* |
| 1 | Gold | 56 | 150 | 40 | 85.7 | −10.6 | 54.5 | 55.5 | 85.7 | −6.7 | 7.7 | 10.2 |
| 2 | Gold | 56 | 180 | 20 | 76.3 | 0.8 | 53.2 | 53.2 | 80.5 | −8.1 | 13.9 | 16.1 |
| 3 | Red | 56 | 150 | 74 | 71.0 | 43.5 | −0.6 | 43.5 | 84.3 | −12.8 | 49.7 | 51.3 |
| 4 | Red | 56 | 320 | 90 | 70.9 | 42.5 | 0.3 | 42.5 | 82.0 | −21.0 | 32.0 | 38.3 |
| 5 | Violet | 56 | 200 | 40 | 59.1 | 60.8 | −48.9 | 78.0 | 78.9 | −1.2 | 33.0 | 33.0 |
| 6 | Violet | 56 | 210 | 30 | 55.1 | 66.3 | −52.8 | 84.8 | 77.0 | −1.4 | 35.6 | 35.6 |
| 7 | Violet | 56 | 225 | 20 | 51.5 | 63.8 | −54.5 | 83.9 | 73.8 | −0.8 | 36.9 | 36.9 |
| 8 | Blue | 56 | 225 | 40 | 62.2 | 0.1 | −51.0 | 51.0 | 71.2 | 27.8 | −4.7 | 28.2 |
| 9 | Blue | 56 | 230 | 35 | 60.4 | 1.7 | −53.3 | 53.3 | 70.3 | 28.4 | −5.0 | 28.8 |
| 10 | Blue | 56 | 240 | 28 | 58.3 | 0.1 | −54.1 | 54.1 | 68.0 | 30.5 | −7.0 | 31.3 |
| 11 | Blue | 56 | 250 | 21 | 56.9 | 0.4 | −52.5 | 52.5 | 66.2 | 30.2 | −6.9 | 31.0 |
| 12 | Turquoise | 56 | 225 | 54 | 72.5 | −30.6 | −31.0 | 43.6 | 68.4 | 37.1 | −18.1 | 41.3 |
| 13 | Turquoise | 56 | 240 | 40 | 71.2 | −34.3 | −33.5 | 47.9 | 65.6 | 40.8 | −23.6 | 47.1 |
| 14 | Turquoise | 56 | 250 | 32 | 69.5 | −35.9 | −34.4 | 49.7 | 63.2 | 44.1 | −27.1 | 51.8 |
| 15 | Turquoise | 56 | 260 | 25 | 67.1 | −34.7 | −34.8 | 49.1 | 61.1 | 45.7 | −28.5 | 53.9 |
| 16 | Green | 56 | 190 | 93 | 64.7 | −54.7 | 0.1 | 54.7 | 63.5 | 42.9 | −13.7 | 45.0 |
| 17 | Green | 56 | 200 | 88 | 69.4 | −53.3 | −0.4 | 53.3 | 63.1 | 43.2 | −18.6 | 47.0 |
| 18 | Green | 56 | 210 | 83 | 74.1 | −50.1 | 0.5 | 50.1 | 62.9 | 42.3 | −24.6 | 48.9 |
| 19 | Green | 56 | 225 | 75 | 79.7 | −43.3 | 2.2 | 43.4 | 63.0 | 37.9 | −32.4 | 49.9 |

¹±5 nm
²Normal incident hue. The hue of the interference color resulting from a viewing angle which is perpendicular to the plane of the drawdown card, and in which the incident light upon the drawdown card is also from the perpendicular or near it.

Liquid Foundation

| PHASE | INGREDIENTS | WT % |
|---|---|---|
| A. | Xanthan Gum (Keltrol T) | 0.20 |
| | Cellulose Gum (CMC 7LF) | 0.20 |
| | DI Water (q.s. to 100%) | 70.00 |
| B. | Triethanolamine (TEA 99%) | 0.65 |
| | PEG-7 Glyceryl Cocoate (Cetiol HE) | 6.00 |
| | Preservative (Water Soluble) | q.s. |
| C. | Talc | 0.75 |
| | Iron Oxides | 1.20 |
| D. | Multi-quadrant interference effect materials | 5.00 |
| | (Calcium Sodium Borosilicate (and) | |
| | Titanium Dioxide (and) Silica) | |
| | of Ex. 4, 5, 8, 12, and/or 19 in | |
| | Example 6 above | |
| E. | Isopropyl Myristate | 2.00 |
| | Oleyl Alcohol (Novol) | 6.50 |
| | Mineral Oil (and) Lanolin Alcohol | 4.50 |
| | (Americhol L-101) | |
| | Cetearyl Alcohol (Lanette O) | 2.00 |
| | Stearic Acid | 1.00 |
| | Preservatives (Oil Soluble) | q.s. |
| | | 100.0 |

Procedure:

Xanthan Gum (Keltrol T) made by J.M. Huber Corporation, and Cellulose Gum (CMC 7LF) made by Hercules Incorporated, both of Phase A, were dispersed into deionized water using high shear mixing until the mixture was smooth. Phase B ingredients, triethanolamine made by Dow Chemical Corporation, PEG-7 Glyceryl Cocoate (Cetiol HE) made by Cognis Corporation and a water soluble preservative, were added to the smooth gum mixture of Phase A and then mixed until smooth. The ingredients of Phase C, talc and iron oxides, were pulverized and added to the mixture using high shear mixing until the joined components were smooth.

In a support vessel the following ingredients, isopropyl myristate, oleyl alcohol (Novol) made by Croda, Inc., mineral oil (and) lanolin alcohol (Americhol L-101) made by Dow Chemical Corporation, cetearyl alcohol (Lanette O) made by Cognis Corporation, stearic acid and oil soluble preservatives were heated to 75+/−5 degrees C. with gentle agitation.

The multi-quadrant interference effect material of Samples 4, 5, 8, 12, and/or 19 of Example 6 above were added to the Phase A-B-C mixture with gentle agitation, and maintained at a temperature of 75+/−5 degrees C. The components heated in the support vessel were added to the Phase A-B-C and multi-quadrant interference effect material mixture with gentle agitation, maintaining a temperature at 75+/−5 degrees C. A constant agitation was maintained and the overall mixture was cooled to 35+/−5 degrees C.

The opacity of the liquid foundation base reduced the intensity of the colors. However, when the liquid foundation makeup is applied on the skin, we are able to observe the following result for skin appearance: With non-quarter wave blue (Sample #8), the color changes from orange, red, purple, blue, turquoise, depending on the viewing angle. With non-quarter wave red (Sample #4), the color changes from gold, orange, red, violet, depending on the viewing angle. With non-quarter wave turquoise (Sample #12), the color changes from red, purple, blue, turquoise, depending on the viewing angle. With non-quarter wave violet (Sample #5), the color changes from orange, red, purple, blue, depending on the viewing angle. With non-quarter-wave green (Sample #19), the color changes from purple, blue, turquoise, green, depending on the viewing angle.

### EXAMPLE 8

Pressed Powder Eye Shadow

| PHASE | INGREDIENTS | WT % |
|---|---|---|
| A. | Talc (q.s. to 100%) | 49.50 |
| | Mearlmica ® SVA (Mica (and) Lauroyl Lysine) | 10.00 |
| | Magnesium Myristate | 5.00 |
| | Silica (Spherica P-1500) | 2.00 |
| | Chroma-Lite ® Brown CL4509 | 0.50 |
| | (Mica (and) Bismuth Oxychloride | |
| | (and) Iron Oxides) | |
| B. | Multi-quadrant interference effect materials | 15.00 |
| | (Calcium Sodium Borosilicate (and) | |
| | Titanium Dioxide (and) Silica) | |
| | of Samples 4, 5, 8, 12, and/or 19 | |
| | in Example 6 above | |
| | Preservatives | q.s. |
| C. | Octyl Palmitate (Ceraphyl 368) | 7.00 |
| | Isostearyl Neopentanoate (Ceraphyl 375) | 1.00 |
| | Antioxidants | q.s. |
| D. | Multi-quadrant interference effect materials | 10.00 |
| | (Calcium Sodium Borosilicate (and) | |
| | Titanium Dioxide (and) Silica) | |
| | of Samples 4, 5, 8, 12, and/or 19 | |
| | in Example 6 above | |
| | | 100.00 |

Procedure:

In an appropriate dry blending/dispersing equipment, talc, Mearlmica® SVA (Mica (and) Lauroyl Lysine) made by the Engelhard Corporation, magnesium myristate, silica (Spherica P-1500) made by the Ikeda Corporation, and Chroma-Lite® Brown CL4509 also made by the Engelhard Corporation, were thoroughly blended and dispersed. Multi-quadrant interference effect material of Samples 4, 5, 8, 12, and/or 19 in Example 6 above and preservatives were added to the dry blended ingredients and mixed until uniform.

Octyl palmitate (Ceraphyl 368) made by ISP, isostearyl neopentanoate (Ceraphyl 375) also made by ISP and antioxidants were added to a support vessel and heated and mixed until uniform.

Next, the Ceraphyl 368, Ceraphyl 375 and antioxidant mixture was sprayed into the premixed dry blended ingredients and effect pigment mixture following which blending of the total composition was resumed. The ingredients were removed from the dry blending/dispersing equipment, pulverized and then returned to the blender. After which point, additional multi-quadrant interference effect material of Samples 4, 5, 8, 12, and/or 19 above were added to the total composition and mixed until uniform.

When the opaque pressed powder eye shadow containing goniochromatic non-quarter-wave multi-quadrant interference color travel effect pigment is applied onto the skin, we observed the following strong color travel results: With non-quarter wave blue (Sample #8), the color changes from orange, red, purple, blue, turquoise, depending on the viewing angle. With non-quarter wave red (Sample #4), the color changes from gold, orange, red, violet, depending on the viewing angle. With non-quarter wave turquoise (Sample #12), the color changes from red, purple, blue, turquoise, depending on the viewing angle. With non-quarter wave violet (Sample #5), the color changes from orange, red, purple, blue, depending on the viewing angle. With non-quarter-wave green (Sample #19), the color changes from purple, blue, turquoise, green, depending on the viewing angle.

### EXAMPLE 9

Nail Enamel

| INGREDIENTS | WT % |
|---|---|
| Suspending Lacquer SLF-2 (Butyl Acetate (and) Toluene (and) Nitrocellulose (and) | 82.00 |
| Tosylamide/Formaldehyde Resin (and) Isopropyl Alcohol (and) Dibutyl Phthalate | |
| (and) Ethyl Acetate (and) Camphor (and) n-Butyl Alcohol (and) Silica | |
| (and) Quaterinum-18 Hectorite) | |
| Multi-quadrant interference effect materials (Calcium Sodium Borosilicate | 3.00 |
| (and) Titanium Dioxide (and) Silica) of Samples 4, 5, 8, 12, and/or 19 in | |
| Example 6 above | |
| Lacquer 127P (Butyl Acetate (and) Toluene (and) Nitrocellulose (and) | 15.00 |
| Tosylamide/Formaldehyde Resin (and) Isopropyl Alcohol (and) | |
| Dibutyl Phthlate (and) Ethyl Acetate (and) Camphor (and) n-Butyl Alcohol)¹ | |
| | 100.00 |

Procedure:

Suspending lacquer SLF-2, multi-quadrant interference effect material of Samples 4, 5, 8, 12,and/or 19 above, and lacquer 127P, all components made by the Engelhard Corporation, were combined into an appropriate size vessel fitted with a Lightnin™ type propeller mixer. The components were mixed until the components were made uniform.

We observed the following strong color travel results both in the clear nail enamel and in the lacquer applied to the nail: With non-quarter wave blue (Sample #8), the color changes from orange, red, purple, blue, turquoise, depending on the viewing angle. With non-quarter wave red (Sample #4), the color changes from gold, orange, red, violet, depending on the viewing angle. With non-quarter wave turquoise (Sample #12), the color changes from red, purple, blue, turquoise, depending on the viewing angle. With non-quarter wave violet (Sample #5), the color changes from orange, red, purple, blue, depending on the viewing angle. With non-quarter-wave green (Sample #19), the color changes from purple, blue, turquoise, green, depending on the viewing angle.

### EXAMPLE 10

Nail Enamel

| INGREDIENTS | WT % |
|---|---|
| Suspending Lacquer SLF-2 (Butyl Acetate (and) Toluene (and) Nitrocellulose (and) | 82.00 |
| Tosylamide/Formaldehyde Resin (and) Isopropyl Alcohol (and) Dibutyl Phthalate | |
| (and) Ethyl Acetate (and) Camphor (and) n-Butyl Alcohol (and) Silica | |
| (and) Quaterinum-18 Hectorite) | |
| Multi-quadrant interference effect materials (Calcium Sodium Borosilicate | 2.85 |
| (and) Titanium Dioxide (and) Silica) of Samples 4, 5, 8, 12, and/or 19 | |
| in Example 6 above | |
| Duocrome ® BR 426C (Mica (and) Titanium Dioxide (and) Ferric Ferrocyanide) | 3.00 |
| Lacquer 127P (Butyl Acetate (and) Toluene (and) Nitrocellulose (and) | 12.15 |
| Tosylamide/Formaldehyde Resin (and) Isopropyl Alcohol (and) | |
| Dibutyl Phthlate (and) Ethyl Acetate (and) Camphor (and) n-Butyl Alcohol) | |
| | 100.00 |

Procedure:

Suspending ISLF-2, multi-quadrant interference effect material of Samples 4, 5, 8, 12, and/or 19 above, Duocrome® BR 426C pigment and lacquer 127P, all components made by the Engelhard Corporation, were combined into an appropriate size vessel fitted with a Lightnin™ type propeller mixer. The components were continuously mixed until the components were made uniform.

We observed the following strong color travel results in the clear nail enamel. With non-quarter wave blue (Sample #8), the color changes from orange, red, purple, blue, turquoise, depending on the viewing angle. With non-quarter wave red (Sample #4), the color changes from gold, orange, red, violet, depending on the viewing angle. With non-quarter wave turquoise (Sample #12), the color changes from red, purple, blue, turquoise, depending on the viewing angle. With non-quarter wave violet (Sample #5), the color changes from orange, red, purple, blue, depending on the viewing angle. With non-quarter-wave green (Sample #19), the color changes from purple, blue, turquoise, green, depending on the viewing angle.

### EXAMPLE 11

Lipstick

| PHASE | INGREDIENTS | WT % |
|---|---|---|
| A. | Candelilla Wax | 2.75 |
| | Carnauba Wax | 1.25 |
| | Beeswax | 1.00 |
| | Ceresine Wax | 5.90 |
| | Ozokerite Wax | 6.75 |
| | Microcrystalline Wax (Multiwax 180W) | 1.40 |
| | Oleyl Alcohol (Novol) | 3.00 |
| | Isostearyl Palmitate (Jeechem ISP) | 7.50 |
| | Isostearyl Isostearate (Schercemol 1818) | 5.00 |
| | Bis-Diglycerylpolyalcohol Adipate (Sofyisan 649) | 2.00 |
| | Acetylated Lanolin Alcohol (Acetulan) | 2.50 |
| | Sorbitan Tristearate (Crill 35) | 2.00 |
| | Aloe Vera (Veragel Lipoid 1:1) | 1.00 |
| | Castor Oil (q.s. to 100%) | 42.80 |
| | Red 7 Lake | 0.10 |
| | Tocopheryl Acetate | 0.20 |
| | Antioxidant | q.s. |
| | Preservatives | q.s. |
| B. | Multi-quadrant interference effect materials | 14.75 |
| | (Calcium Sodium Borosilicate (and) Titanium | |
| | Dioxide (and) Silica) | |
| | of Samples 4, 5, 8, 12, and/or 19 in | |
| | Example 6 above | |
| C. | Fragrance | 0.10 |
| | | 100.00 |

Procedure:

Phase A ingredients, candelilla wax, carnauba wax, beeswax, ceresine wax, ozokerite wax, Microcrystalline Wax (Multiwax 180W) made by Crompton Corporation, Oleyl Alcohol (Novol) made by Croda, Inc., Isostearyl Palmitate (Jeechem ISP) made by Jeen International Corporation, Isostearyl Isostearate (Schercemol 1818) made by Noveon, Inc., Bis-Diglycerylpolyalcohol Adipate-2 (Sofyisan 649) made by Sasol North America, Inc., Acetylated Lanolin Alcohol (Acetulan) made by Dow Chemical Company, Sorbitan Tristearate (Crill 35) made also by Croda, Inc., Aloe Vera (Veragel Lipoid 1:1) made by Pureworld Botanicals, Inc., castor oil, red 7 lake, tocopheryl acetate, antioxidant and preservatives, were all weighed and placed into a heated vessel with the temperature being raised to 85+/−3 degrees C. The ingredients were stirred until they are melted and uniform.

To the Phase A ingredients, multi-quadrant interference effect material of Samples 4, 5, 8, 12, and/or 19 of example 6 above were added and mixed until all the pearl pigment were well dispersed. Fragrance was then added and mixed with constant stirring. The composition was poured at 75+/− degrees C. Finally, the composition was molded, cooled and flamed into the lipsticks. When iron oxide or organic pigments are used, they should first be dispersed in castor oil; this mixture should then be milled in either a colloid or roller mill.

We observed the following result in the opaque lipstick: With non-quarter wave blue (Sample #8), the color changes from orange, red, purple, blue, turquoise, depending on the viewing angle. With non-quarter wave red (Sample #4), the color changes from gold, orange, red, violet, depending on the viewing angle. With non-quarter wave turquoise (Sample #12), the color changes from red, purple, blue, turquoise, depending on the viewing angle. With non-quarter wave violet (Sample #5), the color changes from orange, red, purple, blue, depending on the viewing angle. With non-quarter-wave green (Sample #19), the color changes from purple, blue, turquoise, green, depending on the viewing angle.

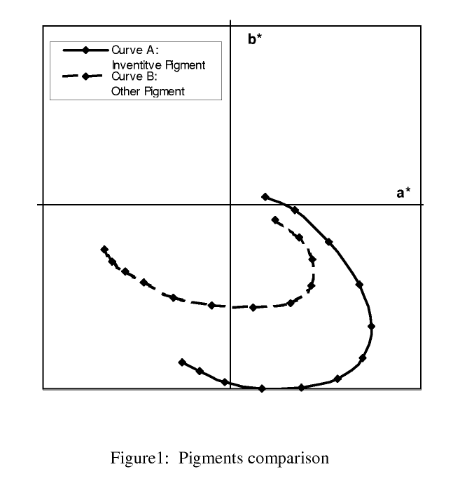

In addition to the visual observations, both lipstick containing non-quarter-wave blue (Sample #8) and traditional quarter-wave blue were applied to a drawdown card. The color travel effects were measured by a Goniospectrophotometer (CMS-1500) manufactured by Murakami Color Research Laboratory for Hunter Associates Laboratory Inc., Reston, Va. Referring to FIG. 1, the graph compares the color travel of a pigment of this invention, a non-quarter wave blue, (Curve A) with a commercially available traditional quarter-wave blue pigment (Curve B). As shown in FIG. 1, the color travel of Curve A travels further than Curve B by having passed through three quadrants. The strong color travel and intense chroma of the non-quarter-wave blue pigment results in the opaque based lipstick containing the non-quarter-wave blue pigment having a stronger color travel and more chroma than lipstick containing a commercially available quarter-wave blue pigment.

### EXAMPLE 12

Hair Shampoo

| PHASE | INGREDIENTS | WT % |
|---|---|---|
| A. | DI Water (q.s. to 100%) | 69.80 |
| | Polyquaternium-4 (Celquat H-100) | 0.50 |
| | Acrylates/Aminoacrylates Copolymer | 8.00 |
| | (Structure Plus) | |
| B. | Sodium Laureth Sulfate (Jeelate ES-270) | 15.00 |
| | Cocamidopropyl Betaine (Jeeteric CAB-LC) | 4.00 |
| | Linoleamidopropyl PG-Dimonium Chloride | 1.00 |
| | Phosphate Dimethicone (Arlasikl | |
| | Phospholipid PLN)³ | |
| C. | Citric Acid (25% aqueous solution) (q.s. to pH 6.0) | q.s. |
| | Preservative | q.s. |
| | UV stabilizer | q.s. |
| D. | Fragrance (Fruity Floral 11301V) | 0.20 |
| | Antioxidant | q.s. |
| | Polysorbate 20 (Tween 20) | 0.40 |
| E. | DI Water | 1.00 |
| | Multi-quadrant interference effect materials | 0.10 |
| | (Calcium Sodium Borosilicate (and) | |
| | Titanium Dioxide (and) Silica) | |
| | of Samples 4, 5, 8, 12, and/or 19 | |
| | in Example 6 above | |
| | | 100.00 |

Procedure:

Phase A ingredients, Polyquaternium-4 (Celquat H-100) made by National Starch and Chemical Company was added to deionized water having used moderate propeller agitation and was mixed at room temperature. The Celquat H-100 and water combination was then heated to 50 degrees C. and to it acrylates/aminoacrylates copolymer (Structure Plus) was added while mixing.

Next, Phase B ingredients, Sodium Laureth Sulfate (Jeelate ES-270) made by Jeen International Corporation, Cocamidopropyl Betaine (Jeeteric CAB-LC) also made by Jeen International Corporation, and Linoleamidopropyl PG-Dimonium Chloride Phosphate Dimethicone (Arlasikl Phospholipid PLN) made by Uniqema, were added in the listed order at 50 degrees C. with proper mixing. Care was taken so that aeration was avoided. The Phase A-B composition was then cooled down to 40 degrees C. to which then Phase C ingredients, citric acid, preservative, and UV stabilizer, were individually added.

Phase D ingredients fragrance (Fruity Floral 11301V) made by Shaw Mudge & Company and antioxidant were pre-mixed. In some cases, slight heat may be needed to attain uniformity, though the heat should not exceed 40-45 degrees C. Once the fragrance and anti-oxidant composition was uniform, it was cooled to 25 degrees C. and Polysorbate 20 (Tween 20) made by Uniqema was added to the pre-mix. The pre-mixed components of Phase D were then added to the Phase A-B-C composition and mixed until uniform.

Phase E ingredients, deionized water was pre-mixed with multi-quadrant interference effect material of Samples 4, 5, 8, 12, and/or 19 in Example 6 above, and then added to the Phase A-B-C-D composition with good mixing. The Phase A-B-C-D-E composition was dropped once it was uniform.

We observed the following strong color travel result in the clear hair shampoos: With non-quarter wave blue (Sample #8), the color changes from orange, red, purple, blue, turquoise, depending on the viewing angle. With non-quarter wave red (Sample #4), the color changes from gold, orange, red, violet, depending on the viewing angle. With non-quarter wave turquoise (Sample #12), the color changes from red, purple, blue, turquoise, depending on the viewing angle. With non-quarter wave violet (Sample #5), the color changes from orange, red, purple, blue, depending on the viewing angle. With non-quarter-wave green (Sample #19), the color changes from purple, blue, turquoise, green, depending on the viewing angle.

### EXAMPLE 13

Hair Pomade

| PHASE | INGREDIENTS | WT % |
|---|---|---|
| A. | White Petrolatum (Super White Protopet) | 46.20 |
| | (q.s. to 100%) | |
| | Glyceryl Dilaurate (Emulsynt GDL) | 30.00 |
| | Octodecyl Stearoyl Stearate (Ceraphyl 847) | 10.00 |
| | PEG-20 Stearate (Cerasynt 840) | 5.00 |
| | Maleated Soybean Oil (Ceraphyl NGA) | 5.00 |
| | Lauryl Lactate (Ceraphyl 31) | 3.00 |
| | Corn(Zea Mays) Oil (and) BHA (and) BHT (Tenox-4) | 0.10 |
| | Preservatives (oil soluble) | q.s. |
| | Benzophenone-3 (Escalol 567) | 0.50 |
| B. | Fragrance (21325G) | q.s. |
| | Multi-quadrant interference effect materials | 0.20 |
| | (Calcium Sodium Borosilicate (and) Titanium Dioxide | |
| | (and) Silica) of Samples 4, 5, 8, 12, and/or 19 | |
| | in Example 6 above | |
| | | 100.00 |

Procedure:

Phase A ingredients, White Petrolatum (Super White Protopet) made by Crompton Corporation, Glyceryl Dilaurate (Emulsynt GDL) made by ISP, Octodecyl Stearoyl Stearate (Ceraphyl 847) also made by ISP, PEG-20 Stearate (Cerasynt 840) made by ISP, maleated soybean oil (Ceraphyl NGA), Lauryl Lactate (Ceraphyl 31) made by ISP, Corn (Zea Mays) Oil (and) BHA (and) BHT (Tenox-4) made by Eastman Chemical Company, oil soluble preservatives, and Benzophenone-3 (Escalol 567) made by ISP, were weighed and added into a heated vessel. The temperature of the vessel was raised to 59+/−3 degrees C. and the ingredients were stirred until melted and uniform. Phase B ingredients fragrance (21325G) made by Shaw Mudge & Company and multi-quadrant interference effect material of Samples 4, 5, 8, 12, and/or 19 above were pre-mixed and then added to the melted and uniform ingredients of Phase A. The composition was mixed until all the pigments were well dispersed. Finally, the composition was poured at 40+/−3 degrees C.

We observed the following result in the hair pomade: With non-quarter wave blue (Sample #8), the color changes from orange, red, purple, blue, turquoise, depending on the viewing angle. With non-quarter wave red (Sample #4), the color changes from gold, orange, red, violet, depending on the viewing angle. With non-quarter wave turquoise (Sample #12), the color changes from red, purple, blue, turquoise, depending on the viewing angle. With non-quarter wave violet (Sample #5), the color changes from orange, red, purple, blue, depending on the viewing angle. With non-quarter-wave green (Sample #19), the color changes from purple, blue, turquoise, green, depending on the viewing angle.

### EXAMPLE 14

Body Splash

| PHASE | INGREDIENTS | WT % |
|---|---|---|
| A. | DI Water (q.s. to 100%) | 28.33 |
| | Disodium EDTA | 0.02 |
| | Acrylates/C10-30 Alkyl Acrylate Cosspolymer | 10.00 |
| | (Carbopol ETD 2020) | |
| | (2% aqueous dispersion) | |
| | 2-Amino 2-Methyl Propanol (AMP-95) | 0.10 |
| | Glycerin (and) Glyceryl Polyacrylate | 2.00 |
| | (Hispagel Oil, Low Viscosity) | |
| B. | Fragrance (Marigold Fragrance C2830) | 0.50 |
| | Polysorbate 20 (and) PEG 40 Castor Oil | 1.00 |
| | (Protachem Solubilizer Blend) | |
| | Glycereth-26 (Protachem GL-26) | 1.00 |
| | Methylpropanediol (MP Diol) | 2.00 |
| C. | Alcohol (SD 39C) | 55.00 |
| | Multi-quadrant interference effect materials | 0.05 |
| | (Calcium Sodium Borosilicate (and) Titanium | |
| | Dioxide (and) Silica) of Samples 4, 5, 8, 12, | |
| | and/or 19 in Example 6 above | |
| | | 100.00 |

Procedure:

Phase A ingredients, disodium EDTA, acrylates/C10-30 Alkyl Acrylate Crosspolymer (Carbopol ETD 2020) made by Noveon, Inc., 2-Amino 2-Methyl Propanol (AMP-95) made by Dow Chemical Company, and Glycerin (and) Glyceryl Polyacrylate (Hispagel Oil, Low Viscosity) made by Hispano Quimica S.A./Centerchem, Inc. were added in order to deionized water at room temperature with moderate agitation. The composition was mixed until uniform, and aeration was avoided.

Phase B ingredients, fragrance (Marigold Fragrance C2830) made by Carrubba, Inc., Polysorbate 20 (and) PEG 40 Castor Oil (Protachem Solubilizer Blend) made by Protameen Chemicals, Inc., Glycereth-26 (Protachem GL-26) also made by Protameen Chemicals, Inc., and Methylpropanediol (MP Diol) made by Lyondell Chemical Company, were pre-mixed at room temperature. Separately, Phase C ingredients alcohol (SD 39C) and multi-quadrant interference effect material of Samples 4, 5, 8, 12, and/or 19 in Example 6 above were premixed at room temperature. The separately premixed Phase B and Phase C were combined and then added to the uniform mixture of Phase A with moderate agitation. The composition was mixed until uniform and aeration was avoided.

We observed the following strong color travel result in the clear body splash: With non-quarter wave blue (Sample #8), the color changes from orange, red, purple, blue, turquoise, depending on the viewing angle. With non-quarter wave red (Sample #4), the color changes from gold, orange, red, violet, depending on the viewing angle. With non-quarter wave turquoise (Sample #12), the color changes from red, purple, blue, turquoise, depending on the viewing angle. With non-quarter wave violet (Sample #5), the color changes from orange, red, purple, blue, depending on the viewing angle. With non-quarter-wave green (Sample #19), the color changes from purple, blue, turquoise, green, depending on the viewing angle.

### EXAMPLE 15

Hair Gel

| PHASE | INGREDIENTS | WT % |
|---|---|---|
| A. | Water (q.s. to 100%) | 79.20 |
| | Carbomer (Carbopol Ultrez 10) | 0.50 |
| B. | Propylene Glycol | 4.00 |
| | Preservative | q.s. |
| C. | Neutralizing agent | 0.50 |
| D. | Water | 15.00 |
| | PVP | 0.20 |
| | Disodium EDTA | 0.05 |
| | Benzophenone-4 (Uvinul MS-40) | 0.05 |
| E. | Polysorbate 20 (Tween 20) | 0.20 |
| | Fragrance (CK TYPE#18567H) | 0.10 |
| | Multi-quadrant interference effect materials | 0.20 |
| | (Calcium Sodium Borosilicate (and) Titanium | |
| | Dioxide (and) Silica) of Samples 4, 5, 8, 12, | |
| | and/or 19 in Example 6 above | |
| | | 100.00 |

Procedure:

Ingredients of Phase A, water and Carbomer (Carbopol Ultrez 10) made by Noveon, Inc., were combined and mixed until thoroughly dispersed. Phase B ingredients, propylene glycol and preservative, were premixed then added to the Phase A ingredients with continued mixing until the composition was completely uniform. Under agitation, neutralizing agent was added to the Phase A and the pre-mixed Phase B combination. Phase C ingredients water, PVP, Disodium EDTA, and Benzophenone-4 (Uvinul MS-40) made by the BASF Corporation were pre-mixed until dissolved and then added to the Phase A-B and neutralizing agent combination. Phase D ingredients, Polysorbate 20 (Tween 20) made by Uniqema, fragrance (CK TYPE#18567H) made by Shaw Mudge & Company, and multi-quadrant interference effect material of Samples 4, 5, 8, 12, and/or 19 in Example 6 above were pre-mixed and then add to the Phase A-B-C and triethanolamine combination.

We observed the following strong color travel result in the clear hair gel: With non-quarter wave blue (Sample #8), the color changes from orange, red, purple, blue, turquoise, depending on the viewing angle. With non-quarter wave red (Sample #4), the color changes from gold, orange, red, violet, depending on the viewing angle. With non-quarter wave turquoise (Sample #12), the color changes from red, purple, blue, turquoise, depending on the viewing angle. With non-quarter wave violet (Sample #5), the color changes from orange, red, purple, blue, depending on the viewing angle. With non-quarter-wave green (Sample #19), the color changes from purple, blue, turquoise, green, depending on the viewing angle.

### EXAMPLE 16

Deodorant Stick

| PHASE | INGREDIENTS | WT % |
|---|---|---|
| A. | DI Water (q.s. to 100%) | 18.50 |
| | Propylene Glycol | 58.20 |
| | Castor Oil/IPDI Copolymer | 12.00 |
| | (Polyderm PPI-CO-200) | |
| | Sodium Stearate (Sodium Stearate C-7) | 8.00 |
| | Isostearth-2 Alcohol (Dermocol IS-2) | 2.00 |
| | UV Absorbers | q.s. |
| B. | Multi-quadrant interference effect materials | 0.10 |
| | (Calcium Sodium Borosilicate (and) Titanium Dioxide | |
| | and) Silica) of Samples 4, 5, 8, 12, and/or 19 | |
| | in Example 6 above | |
| C. | DI Water | 1.00 |
| | Actysse premiere BG100 | 0.20 |
| | (Calcium Sodium Phosphosilicate (and) Mica) | |
| | | 100.00 |

Procedure:

Phase A ingredients, propylene glycol was mixed with deionized water and heated to 80-85 degrees C. Then, Castor Oil/IPDI Copolymer (Polyderm PPI-CO-200) made by Alzo International Inc. was added in small increments to the water-glycol combination until all was melted and dissolved.

Next, Sodium Stearate (Sodium Stearate C-7) made by Crompton Corporation was added to the water-glycol-Polyderm PPI-CO-200 combination, with the temperature having been maintained at 85 degrees C. until the solution was clear. Finally, the remaining Phase A ingredients Isostearth-2 Alcohol (Dermocol IS-2) and UV absorbers were added to the combination and mixed well.

Phase B ingredients, multi-quadrant interference effect material of Examples 4, 5, 8, 12, and/or 19 in Example 6 above were pre-dispersed and then added to the prepared Phase A ingredients.

Finally, Phase C ingredients, deionized water and Actysse™ premiere BG100 also made by the Engelhard Corporation were added to the Phase A-B combination and mixed until uniform.

We observed the following strong color travel result when the deodorant stick is applied onto skin: With non-quarter wave blue (Sample #8), the color changes from orange, red, purple, blue, turquoise, depending on the viewing angle. With non-quarter wave red (Sample #4), the color changes from gold, orange, red, violet, depending on the viewing angle. With non-quarter wave turquoise (Sample #12), the color changes from red, purple, blue, turquoise, depending on the viewing angle. With non-quarter wave violet (Sample #5), the color changes from orange, red, purple, blue, depending on the viewing angle. With non-quarter-wave green (Sample #19), the color changes from purple, blue, turquoise, green, depending on the viewing angle.

### EXAMPLE 17

Sunscreen Gel

| PHASE | INGREDIENTS | WT % |
|---|---|---|
| A. | DI Water (q.s. to 100%) | 78.75 |
| | Acrylates/C10-30 Alkyl Acrylate Crosspolymer | 0.75 |
| | (Carbopol ETD 2020) | |
| | Multi-quadrant interference effect materials | 1.55 |
| | (Calcium Sodium Borosilicate (and) Titanium Dioxide | |
| | (and) Silica) of Samples 4, 5, 8, 12, and/or 19 | |
| | in Example 6 above | |
| | Tromethamine (Tris Amino) | 1.20 |
| B. | DI water | 10.00 |
| | Tromethamine (Tris Amino) | 2.40 |
| | Phenylbenzimidazole Sulfonic Acid (Parsol HS) | 4.00 |
| | Tetrasodium EDTA (Versene 100) | 0.10 |
| C. | PPG-12-Buteth-16 (Ucon Fluid 50-HB-660) | 0.35 |
| | Preservatives | q.s. |
| | Aloe Vera Gel (Aloe Moist) | 0.50 |
| | Extract of Chamomile (Actiphyte of Chamomile) | 0.10 |
| D. | Fragrance (Coconut NA-74) | 0.10 |
| | Polysorbate-20 (Tween 20) | 0.20 |
| | | 100.00 |

Procedure:

In a first phase (Phase A), Acrylates/C10-30 Alkyl Acrylate Crosspolymer (Carbopol ETD 2020) made by Noveon, Inc. was dispersed in deionized water under constant agitation with aeration having been avoided. Then, multi-quadrant interference effect material of Samples 4, 5, 8, 12, and/or 19 in Example 6 above was added to the Carbopol ETD 2020 and water mixture. When the above listed ingredients of Phase A were dispersed it was partially neutralized with Phase A ingredient Tromethamine (Tris Amino) made by Dow Chemical Company.

Ingredients of Phase B, deionized water, Tromethamine (Tris Amino), Phenylbenzimidazole Sulfonic Acid (Parsol HS) made by DSM Nutritional Products, Inc., and Tetrasodium EDTA (Versene 100), were pre-mixed, heated to 70 degrees C.+/3 degrees C., mixed until clear and subsequently cooled to room temperature. The ingredients of Phase B were then added to the prepared Phase A ingredients and mixed until uniform.

To the Phase A-B combination, Phase C ingredients, PPG-12-Buteth-16 (Ucon Fluid 50-HB-660) made by Dow Chemical Company, preservatives, Aloe Vera Gel (Aloe Moist) made by Protameen Chemicals, Inc. and Extract of Chamomile (Actiphyte of Chamomile) made by Active Organics, Inc., were added in the listed order and mixed until uniform.

Phase D ingredients, fragrance (Coconut NA-74) made by Robertet Fragrances and Polysorbate-20 (Tween 20) made by Uniqema, were pre-mixed then added to the Phase A-B-C combination and mixed until uniform.

We observed the following color travel result in the sunscreen gel: With non-quarter wave blue (Sample #8), the color changes from orange, red, purple, blue, turquoise, depending on the viewing angle. With non-quarter wave red (Sample #4), the color changes from gold, orange, red, violet, depending on the viewing angle. With non-quarter wave turquoise (Sample #12), the color changes from red, purple, blue, turquoise, depending on the viewing angle. With non-quarter wave violet (Sample #5), the color changes from orange, red, purple, blue, depending on the viewing angle. With non-quarter-wave green (Sample #19), the color changes from purple, blue, turquoise, green, depending on the viewing angle.

### EXAMPLE 18

Lip Gloss

| PHASE | INGREDIENTS | WT % |
|---|---|---|
| A | Hydrogenated Polyisobutene (and) | 70.50 |
| | Ethylene/Propylene/Styrene Copolymer (and) | |
| | Butylene/Ethylene/Styrene Copolymer (Versagel | |
| | ME-750) | |
| | Preservatives (Oil Soluble) | q.s. |
| | Octyl Palmitate (Jeechem OP) | 12.00 |
| | Tridecyl Neopentanoate (Ceraphyl 55) | 10.00 |
| | Isostearyl Isostearate (Schercemol 1818) | 5.00 |
| B. | Multi-quadrant interference effect materials | 2.50 |
| | (Calcium Sodium Borosilicate (and) Titanium Dioxide | |
| | (and) Silica) of Examples 4, 5, 8, 12, and/or 19 | |
| | in Example 6 above | |
| C. | Fragrance | q.s. |
| | | 100.00 |

Procedure:

Phase A ingredients, Hydrogenated Polyisobutene (and) Ethylene/Propylene/Styrene Copolymer (and) Butylene/Ethylene/Styrene Copolymer (Versagel ME-750), oil soluble preservatives, Octyl Palmitate (Jeechem OP) made by Jeen International Corporation, Tridecyl Neopentanoate (Ceraphyl 55) made by ISP, and Isostearyl Isostearate (Schercemol 1818) made by Noveon, Inc., were weighed and introduced into a heated vessel. The temperature of the vessel was raised to 70-75 degrees C., and the Phase A ingredients were stirred until the composition was melted and uniform. To the Phase A ingredients, Phase B ingredient multi-quadrant interference effect material of Samples 4, 5, 8, 12, and/or 19 in Example 6 above were added while the temperature of 70-75 degrees C. was maintained. Then to the Phase A-B combination, fragrance was added and mixed under constant stirring.

We observed the following strong color travel result in the clear lip gloss: With non-quarter wave blue (Sample #8), the color changes from orange, red, purple, blue, turquoise, depending on the viewing angle. With non-quarter wave red (Sample #4), the color changes from gold, orange, red, violet, depending on the viewing angle. With non-quarter wave turquoise (Sample #12), the color changes from red, purple, blue, turquoise, depending on the viewing angle. With non-quarter wave violet (Sample #5), the color changes from orange, red, purple, blue, depending on the viewing angle. With non-quarter-wave green (Sample #19), the color changes from purple, blue, turquoise, green, depending on the viewing angle.

### EXAMPLE 19

Lotion

| PHASE | INGREDIENTS | WT % |
|---|---|---|
| A. | Water (q.s. to 100%) | 66.60 |
| | Stearamidopropyl PG-Dimonium Chloride | |
| | Phosphate (and) Cetyl Alcohol | 2.00 |
| | (Arasilk ™ Phospholipid SV) | |
| B. | Butylene Glycol | 4.00 |
| | Preservatives | q.s. |
| C. | Isostearyl Palmitate (Jeechem ISP) | 3.00 |
| | Petrolatum | 3.00 |
| | Ceteareth 20 (and) Cetearyl Alcohol | |
| | (Jeecol CS-20D) | 1.50 |
| D. | Polyquaternium-37 (and) Propylene | 3.00 |
| | Glycol/Dicaprylate Dicaprate (and) PPG-1 | |
| | Trideceth-6 (Salcare SC96) | |
| E. | Water | 1.00 |
| | Preservatives (water soluble) | q.s. |
| F. | DI Water | 10.00 |
| | Tinogard Q (Tris (Tetramethylhydroxypiperidinol) | 0.50 |
| | Citrate) | |
| | Yellow 10 (0.5% aqueous solution) | 0.10 |
| | Green 3 (0.5% aqueous solution) | 0.30 |
| | Multi-quadrant interference effect materials | 5.00 |
| | (Calcium Sodium Borosilicate (and) Titanium | |
| | Dioxide (and) Silica) of Samples 4, 5, 8, 12, | |
| | and/or 19 in Example 6 above | |
| | | 100.00 |

Procedure:

Using a moderate propeller agitation, Phase A ingredients Stearamidopropyl PG-Dimonium Chloride Phosphate (and) Cetyl Alcohol (Arasilk™ Phospholipid SV) made by Uniqema was added to water and heated to 70 degrees C. Phase B ingredients butylene glycol and preservatives were pre-mixed and added, by mixing, to the Phase A ingredients.

Phase C ingredients, Isostearyl Palmitate (Jeechem ISP) made by Jeen International Corporation, petrolatum, and Ceteareth 20 (and) Cetearyl Alcohol (Jeecol CS-20D) made by Jeen International Corporation, were heated to 70 degrees C. and then added to the Phase A and the Phase B combination. The Phase A-B-C combination was then cooled to 40 degrees C. Next, Phase D ingredient Polyquaternium-37 (and) Propylene Glycol/Dicaprylate Dicaprate (and) PPG-1 Trideceth-6 (Salcare SC96), made by CIBA Specialty Chemicals, was added to the Phase A-B-C combination and mixed until uniform. Next, Phase E ingredients, water was pre-mixed with water soluble preservatives and then added, by mixing, to the Phase A-B-C-D combination. Phase F ingredients deionized water, Tinogard Q (Tris (Tetramethylhydroxypiperidinol) Citrate) made by CIBA Specialty Chemicals, yellow 10, green 3, and multi-quadrant interference effect material of Samples 4, 5, 8, 12, and/or 19 above were pre-mixed and added, by mixing, to the Phase A-B-C-D-E combination. The mixing was stopped at 35 degrees C.

We observed the following strong color travel result in the translucent lotion: With non-quarter wave blue (Sample #8), the color changes from orange, red, purple, blue, turquoise, depending on the viewing angle. With non-quarter wave red (Sample #4), the color changes from gold, orange, red, violet, depending on the viewing angle. With non-quarter wave turquoise (Sample #12), the color changes from red, purple, blue, turquoise, depending on the viewing angle. With non-quarter wave violet (Sample #5), the color changes from orange, red, purple, blue, depending on the viewing angle. With non-quarter-wave green (Sample #19), the color changes from purple, blue, turquoise, green, depending on the viewing angle.

### EXAMPLE 20

Lip Gloss

| PHASE | INGREDIENTS | WT % |
|---|---|---|
| A | Hydrogenated Polyisobutene (and) | 70.50 |
| | Ethylene/Propylene/Styrene Copolymer (and) | |
| | Butylene/Ethylene/Styrene Copolymer (Versagel | |
| | ME-750)¹ | |
| | Preservatives (Oil Soluble) | q.s. |
| | Octyl Palmitate (Jeechem OP) | 11.75 |
| | Tridecyl Neopentanoate (Ceraphyl 55) | 10.00 |
| | Isostearyl Isostearate (Schercemol 1818) | 5.00 |
| B. | Multi-quadrant interference effect materials | 2.50 |
| | (Calcium Sodium Borosilicate (and) Titanium Dioxide | |
| | (and) Silica) of Samples 4, 5, 8, 12, and/or 19 | |
| | in Example 6 above | |
| | Duocrome ® YB 622C (Mica (and) Titanium Dioxide | 0.25 |
| | (and) Silica) | |
| C. | Fragrance | q.s. |
| | | 100.00 |

Procedure:

The ingredients in Phase A, Hydrogenated Polyisobutene (and) Ethylene/Propylene/Styrene Copolymer (and) Butylene/Ethylene/Styrene Copolymer (Versagel ME-750) made by Penreco, oil soluble preservatives, Octyl Palmitate (Jeechem OP) made by Jeen International Corporation, Tridecyl Neopentanoate (Ceraphyl 55) made by ISP and Isostearyl Isostearate (Schercemol 1818) made by Noveon, Inc., were weighed and introduced into a heated vessel, with the temperature of the vessel having been raised to 70-75 degrees C. The ingredients were stirred until melted and uniform.

Phase B ingredients, multi-quadrant interference effect material of Samples 4, 5, 8, 12, and/or 19 in Example 6 above and Duocrome® YB 622C made by the Engelhard Corporation were added to the Phase A ingredients under a maintained temperature of 70-75 degrees C. Finally, fragrance was added and mixed into the Phase A-B composition while under constant stirring.

We observed the following strong color travel result in the clear lip gloss: With non-quarter wave blue (Sample #8), the color changes from orange, red, purple, blue, turquoise, depending on the viewing angle. With non-quarter wave red (Sample #4), the color changes from gold, orange, red, violet, depending on the viewing angle. With non-quarter wave turquoise (Sample #12), the color changes from red, purple, blue, turquoise, depending on the viewing angle. With non-quarter wave violet (Sample #5), the color changes from orange, red, purple, blue, depending on the viewing angle. With non-quarter-wave green (Sample #19), the color changes from purple, blue, turquoise, green, depending on the viewing angle.

### EXAMPLE 21

Pressed Powder Hi-Lite

| PHASE | INGREDIENTS | WT % |
|---|---|---|
| A. | Mearltalc ® TCA (Talc (and) Lauroyl Lysine) | 62.40 |
| | (q.s. to 100%) | |
| | Zinc Stearate | 2.10 |
| | Nylon-12 (Orgasol 2002 Natural Cosmetic) | 6.20 |
| | Silk Powder (Crosik Powder) | 1.10 |
| | Multi-quadrant interference effect materials | 15.00 |
| | (Calcium Sodium Borosilicate (and) Titanium Dioxide | |
| | (and) Silica) of Examples 4, 5, 8, 12, and/or 19 | |
| | in Example 6 above | |
| | Preservatives | q.s. |
| B. | Ethylhexyl Methoxycinnamate (Escalol 557) | 2.30 |
| | Isostearyl Neopentanoate (Ceraphyl 375) | 2.10 |
| | Isocetyl Stearoyl Stearate (Ceraphyl 791) | 2.40 |
| | Coco-Caprylate/Caprate (Cetiol LC)⁵ | 1.40 |
| C. | Multi-quadrant interference effect materials | 5.00 |
| | (Calcium Sodium Borosilicate (and) Titanium Dioxide | |
| | (and) Silica) of Samples 4, 5, 8, 12, and/or 19 above | |
| | | 100.00 |

Procedure:

Phase A ingredients, Mearltalc® TCA (Talc (and) Lauroyl Lysine) made by the Engelhard Corporation, zinc stearate, Nylon-12 (Orgasol 2002 Natural Cosmetic) made by Lipo Chemicals, Inc., Silk Powder (Crosik Powder) made by Croda, Inc., multi-quadrant interference effect material, and preservatives were thoroughly blended and dispersed into dry blending/dispersing equipment.

Phase B ingredients Ethylhexyl Methoxycinnamate (Escalol 557) made by ISP, Isostearyl Neopentanoate (Ceraphyl 375) made by ISP, Isocetyl Stearoyl Stearate (Ceraphyl 791) made by ISP, and Coco-Caprylate/Caprate (Cetiol LC) made by Cognis Corporation, were added into a support vessel, heated and mixed until uniform. The Phase B ingredients were then sprayed into the pre-mixed Phase A ingredients. The Phase A-B combination was then pulverized and returned to the blender. Multi-quadrant interference effect material of Samples 4, 5, 8, 12, and/or 19 in Example 6 above were added to the Phase A-B combination and mixed with low shear agitation until uniform. The final composition was then pressed.

We observed the following strong color travel result when the pressed powder hi-lite is applied onto skin: With non-quarter wave blue (Sample #8), the color changes from orange, red, purple, blue, turquoise, depending on the viewing angle. With non-quarter wave red (Sample #4), the color changes from gold, orange, red, violet, depending on the viewing angle. With non-quarter wave turquoise (Sample #12), the color changes from red, purple, blue, turquoise, depending on the viewing angle. With non-quarter wave violet (Sample #5), the color changes from orange, red, purple, blue, depending on the viewing angle. With non-quarter-wave green (Sample #19), the color changes from purple, blue, turquoise, green, depending on the viewing angle.

### EXAMPLE 22

Soap

| INGREDIENTS | WT % |
|---|---|
| Soy Oil Soap Base (Melt & Pour Glycerin Soap) | 99.601 |
| Multi-quadrant interference effect materials (Calcium Sodium | 0.200 |
| Borosilicate (and) Titanium Dioxide (and) Silica) of Samples | |
| 4, 5, 8, 12, and/or 19 in Example 6 above | |
| Chroma-Lite ® Black CL4498 (Mica (and) Bismuth | 0.199 |
| Oxychloride (and) Iron Oxides) | |
| | 100.000 |

Procedure:

Soy oil soap base (Melt & Pour Glycerin Soap) was weighed in an appropriate size vessel and heated until clear. Multi-quadrant interference effect material of Samples 4, 5, 8, 12, and/or 19 in Example 6 above and Chroma-Lite Black CL4498 made by Engelhard Corporation were added to the base. The composition was mixed until uniform.

We observed the following strong color travel with dimensionality result in the clear soap: With non-quarter wave blue (Sample #8), the color changes from orange, red, purple, blue, turquoise, depending on the viewing angle. With non-quarter wave red (Sample #4), the color changes from gold, orange, red, violet, depending on the viewing angle. With non-quarter wave turquoise (Sample #12), the color changes from red, purple, blue, turquoise, depending on the viewing angle. With non-quarter wave violet (Sample #5), the color changes from orange, red, purple, blue, depending on the viewing angle. With non-quarter-wave green (Sample #19), the color changes from purple, blue, turquoise, green, depending on the viewing angle.

### EXAMPLE 23

Soap

| INGREDIENTS | WT % |
|---|---|
| Soy Oil Soap Base (Melt & Pour Glycerin Soap) | 97.70 |
| Multi-quadrant interference effect materials (Calcium Sodium | 0.20 |
| Borosilicate (and) Titanium Dioxide (and) Silica) of Samples | |
| 4, 5, 8, 12, and/or 19 in Example 6 above | |
| External Violet 2 (0.5% aqueous solution) | 2.00 |
| Fragrance | 0.10 |
| | 100.00 |

Procedure:

Soy oil soap base (Melt & Pour Glycerin Soap) was weighed into an appropriate size vessel and heated until clear. Multi-quadrant interference effect material of Samples 4, 5, 8, 12, and/or 19 in Example 6 above, external violet 2 (0.5% aqueous solution), and fragrance was added to the base under continuous mixing until the components were uniform.

We observed the following strong color travel result in the translucent soap: With non-quarter wave blue (Sample #8), the color changes from orange, red, purple, blue, turquoise, depending on the viewing angle. With non-quarter wave red (Sample #4), the color changes from gold, orange, red, violet, depending on the viewing angle. With non-quarter wave turquoise (Sample #12), the color changes from red, purple, blue, turquoise, depending on the viewing angle. With non-quarter wave violet (Sample #5), the color changes from orange, red, purple, blue, depending on the viewing angle. With non-quarter-wave green (Sample #19), the color changes from purple, blue, turquoise, green, depending on the viewing angle.

### EXAMPLE 24

Soap

| INGREDIENTS | WT % |
|---|---|
| Soy Oil Soap Base (Melt & Pour Glycerin Soap) | 99.80 |
| Multi-quadrant interference effect materials (Calcium Sodium | 0.20 |
| Borosilicate (and) Titanium Dioxide (and) Silica) of Samples | |
| 4, 5, 8, 12, and/or 19 in Example 6 above | |
| | 100.00 |

Procedure:

Soy oil soap base (Melt & Pour Glycerin Soap) was weighed into an appropriate size vessel and heated until clear. Multi-quadrant interference effect materials were added to the base and mixed until the composition was uniform.

We observed the following strong color travel result in the semi-opaque soap: With non-quarter wave blue (Sample #8), the color changes from orange, red, purple, blue, turquoise, depending on the viewing angle. With non-quarter wave red (Sample #4), the color changes from gold, orange, red, violet, depending on the viewing angle. With non-quarter wave turquoise (Sample #12), the color changes from red, purple, blue, turquoise, depending on the viewing angle. With non-quarter wave violet (Sample #5), the color changes from orange, red, purple, blue, depending on the viewing angle. With non-quarter-wave green (Sample #19), the color changes from purple, blue, turquoise, green, depending on the viewing angle.

What is claimed is:

1. A cosmetic composition comprising:
(a) a base formulation; and
(b) a goniochromatic pigment comprising
(i) a transparent substrate,
(ii) an optional coating on said transparent substrate (i),
(iii) a layer of titanium dioxide on said transparent substrate (i) or said optional coating (ii), the thickness of said layer of titanium dioxide being such as to provide a white hue to said substrate;
(iv) a subsequent layer of a low refractive index material on said titanium dioxide layer (iii), said subsequent layer of low refractive index material having a thickness of at least 100 nm to provide a variable pathlength for light dependent on the angle of incidence of light impinging thereon,
(v) an optional coating on said subsequent layer (iv); and
(vi) an outermost layer of a high refractive index material placed on said subsequent layer (iv) or said optional coating (v);
each layer differs in refractive index from any adjacent layer by at least about 0.2 and wherein at least one layer has an optical thickness which is different from all of the other layers, whereby the pigment is not a quarter-wave stack.

2. The cosmetic composition of claim 1 wherein the base formulation is transparent.

3. The cosmetic composition of claim 1 wherein the base formulation is translucent.

4. The cosmetic composition of claim 1 wherein the base formulation is semi-opaque.

5. The cosmetic composition of claim 1 wherein the base formulation is opaque.

6. The cosmetic composition of claim 1 wherein the outermost layer comprising titanium dioxide has a thickness of from about 20 to 100 nm.

7. The cosmetic composition of claim 1 wherein the transparent substrate is a glass.

8. The cosmetic composition of claim 1 wherein the transparent substrate is borosilicate.

9. The cosmetic composition of claim 1 wherein the cosmetic composition has multi-quadrant color travel.

10. A cosmetic composition comprising:
a base formulation;
a goniochromatic pigment; and
a combination pigment, the combination pigment including an interference reflection pigment united to an absorption colorant, the absorption colorant is of a color which is different from the reflection color of the interference pigment or the complement thereof and in which the interference pigment and absorption colorant have the same order of magnitude of color intensity.

11. The cosmetic composition of claim 10 wherein the base formulation is transparent.

12. The cosmetic composition of claim 10 wherein the base formulation is translucent.

13. The cosmetic composition of claim 10 wherein the base formulation is semi-opaque.

14. The cosmetic composition of claim 10 wherein the base formulation is opaque.

15. The cosmetic composition of claim 10 wherein the goniochromatic pigment comprises
(i) a transparent substrate,
(ii) an optional coating on said transparent substrate,
(iii) a layer of titanium dioxide on said transparent substrate (i) or said optional coating (ii), the thickness of said layer of titanium dioxide being such as to provide a white hue to said substrate;
(iv) a subsequent layer of a low refractive index material on said titanium dioxide layer (iii), said subsequent layer of low refractive index material having a thickness of at least 100 nm to provide a variable pathlength for light dependent on the angle of incidence of light impinging thereon,
(v) an optional coating on said subsequent layer (iv), and
(vi) an outermost layer of a high refractive index material placed on said subsequent layer (iv) or said optional coating (v);
each layer differs in refractive index from any adjacent layer by at least about 0.2 and wherein at least one layer has an optical thickness which is different from all of the other layers, whereby the pigment is not a quarter-wave stack.

16. The cosmetic composition of claim 15 wherein said outermost layer comprising titanium dioxide has a thickness of from about 20 to 100 nm.

17. The cosmetic composition of claim 15 wherein the transparent substrate is borosilicate.

18. The cosmetic composition of claim 10 wherein the cosmetic composition has multi-quadrant color travel.

19. The cosmetic composition of claim 1 wherein said base formulation is a liquid, a gel, a solid, a powder or a waxy solid.

20. The cosmetic composition of claim 10 wherein said base formulation is a liquid, a gel, a solid, a powder or a waxy solid.
